(19) United States
(12) Patent Application Publication
(10) Pub. No.: US 2017/0319790 A1
(43) Pub. Date: Nov. 9, 2017

(54) Drug Delivery Device with Air-in-Cartridge Safety Feature

(71) Applicant: Novo Nordisk A/S, Bagsvaerd (DK)

(72) Inventors: Carsten Schau Andersen, Seattle, WA (US); Mads Moeller, Hundested (DK); Michael Svendsmark Hansen, Frederiksberg C (DK); Torkil Filholm, Dyssegaard (DK)

(21) Appl. No.: 15/527,400

(22) PCT Filed: Dec. 2, 2015
(86) PCT No.: PCT/EP2015/078357

§ 371 Date: May 17, 2017

(30) Foreign Application Priority Data
Dec. 3, 2014 (EP) 14195984.1

(51) Int. Cl. A61M 5/315; A61M 5/20; G06F 19/00; A61M 5/24; A61M 5/36; A61M 5/31

(52) U.S. Cl. CPC A61M 5/31561; A61M 5/24; A61M 5/31546; A61M 5/365; A61M 5/20; G06F 19/3456; A61M 2205/0216; A61M 2005/3126; A61M 2205/3331; A61M 2205/3584; A61M 2205/50; A61M 2205/70; A61M 2205/8206; A61M 2205/18

(57) ABSTRACT

A drug delivery device adapted to receive a drug-filled cartridge (10), comprising dose setting means allowing a user to set a desired dose of drug to be expelled, an electronic controller (70,270) adapted to control a motor (51) to move a drive member to thereby expel an amount of drug from a received cartridge corresponding to a set dose, and means for estimating the amount of free air in a loaded cartridge. The controller is adapted to detect an error state if the estimated amount of free air in the cartridge is larger than or equals a given percentage of the amount of drug corresponding to a set dose.

The present invention generally relates to drug delivery devices adapted to be used and operated by a patient on his or her own hand. More specifically the invention relates to motorized drug delivery device.

### BACKGROUND OF THE INVENTION

In the disclosure of the present invention reference is mostly made to the treatment of diabetes by subcutaneous drug delivery, either discrete or continuous, however, this is only an exemplary use of the present invention.

The most common type of durable drug delivery devices adapted to receive a drug filled cartridge and expel a discrete dose of a desired size therefrom are driven by manual means or by a spring energized during dose setting, the cartridge being of the type comprising an axially displaceable piston having an initial proximal position and which is moved distally by a piston rod. Subcutaneous drug delivery takes place via an injection needle arranged in fluid communication with the cartridge. The device may be pen-formed or in the form of a more box-shaped so-called doser. In order to improve convenience, user-friendliness and provide additional features, e.g. detection and storing of expelling data, drug delivery devices have been provided with electrically driven means, typically in the form of an electronically controlled motor driving a piston rod through a gear arrangement, e.g. as shown in U.S. Pat. No. 6,514,230 and US 2011/306927.

Whereas motorized drug delivery devices for treatment of diabetes by discrete injections of e.g. insulin are used relatively rarely, in the field of continuous drug delivery portable motorized drug delivery devises have been used widely for decades. The latter type of devices are generally known as infusion pumps and are normally engineered to very high standards and are correspondingly very expensive. Motorized drug delivery devices may also be in the form of larger non-portable infusion systems as known from e.g. U.S. Pat. No. 4,255,088 and WO 2012/126745 which disclose infusion systems provided with means for detecting air in the drug path.

Although a motorized drug delivery device for discrete injections of drug also has to meet very high safety standards, the cost issue is more important as the relatively inexpensive mechanical drug delivery devices, e.g. of the pen-type, to most users are an acceptable alternative. Correspondingly, to make the higher expense acceptable to the user additional advantages should be offered.

Having regard to the above, it is an object of the present invention to provide a motorized drug delivery device which provides a high degree of user-friendliness in a cost-effective way.

### DISCLOSURE OF THE INVENTION

In the disclosure of the present invention, embodiments and aspects will be described which will address one or more of the above objects or which will address objects apparent from the below disclosure as well as from the description of exemplary embodiments.

Thus, in accordance with a general aspect of the invention a drug delivery device is provided comprising a drug-filled cartridge or a compartment for receiving a drug-filled cartridge in a loaded position, the cartridge comprising an outlet and an axially displaceable piston. The device comprises a drug expelling mechanism comprising a drive member adapted to engage and axially move the piston of a loaded cartridge to thereby expel an amount of drug from the cartridge through the outlet, and a motor for moving the drive member. The device further comprises dose setting means allowing a user to set a desired dose of drug to be expelled. An electronic controller is adapted to control the motor to move the drive member to thereby expel an amount of drug corresponding to a set dose. The device further comprises means for estimating the amount of free air in a loaded cartridge. The controller is adapted to detect an error state if the estimated amount of free air in the cartridge is larger than or equals a given percentage of the amount of drug corresponding to a set dose.

When treating type 2 diabetes it is considered a “major” failure if no insulin of a given dose is injected, however, it may be considered only a “minor” failure if at least some insulin of a given dose is injected. Indeed, this is not the case in the treatment of type 1 diabetes.

Based on the above considerations a drug delivery device adapted to estimate the amount of air in a loaded cartridge and thus the amount of drug, e.g. number of units of insulin, corresponding thereto is provided. This information can be compared with a given dose set by the user. For example, if the percentage is set to 100% then if the set dose is larger or equal to the estimated “air dose” then there is the potential risk of injecting air only, this resulting in an error/alarm condition. By this arrangement the traditional “air shot” step performed by the user to remove any air in the cartridge prior to injecting a dose of drug can be eliminated.

The drug delivery device may be provided with user alert means, e.g. audible, visual or tactile, the electronic controller being adapted to actuate the user alert means when an error state is detected.

In an exemplary embodiment the electronic controller is adapted to prevent a set dose of drug to be expelled when an error state is detected, however, the device may be provided with user input means allowing the user to override the electronic controller to thereby allow the motor to move the drive member corresponding to the set dose. An error condition may also be removed by adjusting the set dose to an allowable dose size.

To detect the amount of free air in a cartridge the drug delivery device may be provided with means for detecting a property value of the drug cartridge, the property value varying with the amount of free air contained in the cartridge, wherein the estimated amount of free air in the cartridge is based at least in part on the detected property value. For example, the detected property value may be related to the pressure in the cartridge. The drug delivery device may comprise a force sensor associated with the drive member, the force sensor providing an output related to the pressure in the cartridge during dose expelling. When the cartridge piston is elastically deformable, the detected property value may be related to deformation of the piston. Alternatively, the detected property value may be the capacitance of the loaded cartridge or a portion of the loaded cartridge. As a further alternative the amount of air may be estimated using light emitters and light sensors, the amount of light detected by the sensors being influenced by the amount of free air in a cartridge.

The given percentage may be in the range of 10%-100%, e.g. 25%, 50%, 75% or 100%. The given percentage may be settable, e.g. by the user's doctor.

In an exemplary embodiment the controller may further be adapted to detect an error state if the estimated amount of drug in the cartridge is smaller than the amount of drug corresponding to the set dose, the estimated amount of drug in the cartridge taking into account the amount of estimated free air in the cartridge.

The dose setting means allowing a user to set a desired dose of drug to be expelled may be provided in a number of ways, e.g. by means of conventional buttons, a dial, a touchscreen, or voice control. The dose setting means may be adapted to store one or more pre-set doses. In such an arrangement the controller will compare the estimated amount of free air in the cartridge with the pre-set dose when e.g. the device is turned and is ready to expel the preset dose.

In an exemplary embodiment the device comprises communication means allowing dose setting data to be received from an external device, and an electronic controller adapted to store received dose setting data, and control the motor to move the drive member to thereby expel an amount of drug corresponding to a set dose. By this arrangement a given amount of drug to be expelled can be set and stored in the drug delivery device by external means, this providing a drug delivery device which is both simple and easy to use when a dose corresponding to a stored value is to be delivered. The concept of setting a dose by external means is described in greater detail in EP 2014/060840 which is hereby incorporated by reference.

The controller may be provided with a clock and be adapted to store dose setting data representing at least two pre-set doses, each pre-set dose being associated with a time period of the day and/or week, the controller being adapted to control the motor to move the drive member to thereby expel a stored dose amount according to the actual time.

In accordance with a further aspect of the invention a method of operating a drug delivery device is provided. The method comprises the steps of (i) providing a drug delivery device having a drug-filled cartridge comprising an outlet and an axially displaceable piston, a drug expelling mechanism comprising a drive member adapted to engage and axially move the piston of a loaded cartridge to thereby expel an amount of drug from the cartridge through the outlet, and a motor for moving the drive member, the drug delivery device further having dose setting means allowing a user to set a desired dose of drug to be expelled, an electronic controller adapted to control the motor to move the drive member to thereby expel an amount of drug corresponding to a set dose, and means for estimating the amount of free air in the cartridge, (ii) estimating the amount of free air in the cartridge, (iii) setting a dose to be expelled, and (iv) detecting an error state if the estimated amount of free air in the cartridge is larger than or equals a given percentage of the amount of drug corresponding to the set dose.

As used herein, the term “drug” is meant to encompass any drug-containing flowable medicine capable of being passed through a delivery means such as a cannula or hollow needle in a controlled manner, such as a liquid, solution, gel or fine suspension. Representative drugs include pharmaceuticals such as peptides (e.g. insulins, insulin containing drugs, GLP-1 containing drugs as well as derivates thereof), proteins, and hormones, biologically derived or active agents, hormonal and gene based agents, nutritional formulas and other substances in both solid (dispensed) or liquid form. In the description of the exemplary embodiments reference will be made to the use of insulin containing drugs. Correspondingly, the term “subcutaneous” infusion is meant to encompass any method of transcutaneous delivery to a subject.

### BRIEF DESCRIPTION OF THE DRAWINGS

In the following the invention will be further described with reference to the drawings, wherein

FIG. 1 shows schematically an embodiment of a drug delivery device,

FIG. 2 shows schematically a drive arrangement for a motorized drug delivery device,

FIG. 3A shows data from a first set-up using a force sensor,

FIG. 3B shows data from a second set-up using a force sensor,

FIG. 4 shows a first embodiment of a drug delivery device platform, and

FIGS. 5 and 6 show a second embodiment of a drug delivery device platform.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. When the term member or element is used for a given component it generally indicates that in the described embodiment the component is a unitary component, however, the same member or element may alternatively comprise a number of sub-components just as two or more of the described components could be provided as unitary components, e.g. manufactured as a single injection moulded part. The term “assembly” does not imply that the described components necessarily can be assembled to provide a unitary or functional assembly during a given assembly procedure but is merely used to describe components grouped together as being functionally more closely related.

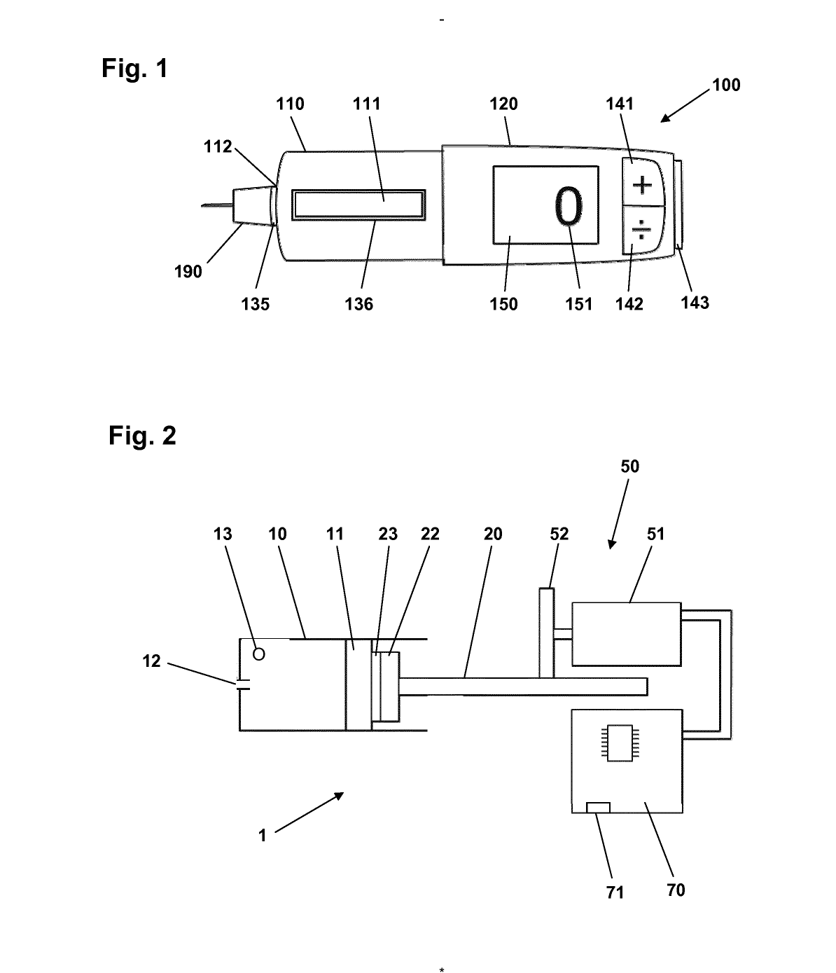

FIG. 1 shows in a schematic representation a generic motorized drug delivery device 100 comprising a main portion 120 in which an expelling assembly is arranged, and a cartridge holder portion 110 adapted to receive and hold an exchangeable drug-filled cartridge. The cartridge holder portion comprises a distal opening 112 and a window 111 allowing a user to visually inspect the content of a loaded cartridge just as the actual position of the cartridge piston can be observed. A needle assembly 190 is mounted in fluid communication with a loaded cartridge. The main portion comprises user input means in the form of a pair of dose setting buttons 141, 142 allowing a user to set and adjust a dose of drug to be expelled, as well as a dose release button 143 arranged at the proximal end of the device. A display 150 shows the currently set dose 151. The display may be controlled to provide further information to a user, e.g. the dose numeral may count down during dose expelling, just as the display may comprises indicators for e.g. battery condition, error conditions, and time.

FIG. 2 shows schematically a drive arrangement for a motorized drug delivery device of the type described with reference to FIG. 1, the arrangement providing a platform for realizing aspects of the present invention.

More specifically, the motorized drug delivery device 1 comprises a main portion in which an expelling assembly 50 is arranged, and a cartridge holder portion adapted to receive and hold an exchangeable drug-filled cartridge 10, the cartridge comprising an axially displaceable piston 11 and a distal outlet 12 associated with coupling means allowing a needle assembly to be mounted. An air bubble 13 is trapped in the cartridge. In the shown embodiment the expelling assembly comprises a drive member in the form of a piston rod 20 adapted to engage and move forward a cartridge piston to thereby expel an amount of drug, the piston rod being driven by an electronically controlled motor 51 via a gear assembly 52. The piston rod comprises a distal piston rod washer 22 in which a sensor 23, e.g. a force sensor, is arranged. The device further comprises electronic controller circuitry 70 adapted to control operation of the motor in order to move the piston rod in a distal or proximal direction, as well as a rechargeable power source (“battery”) associated with the controller circuitry. The electronic controller circuitry may be provided with communication circuitry allowing e.g. pre-set doses to be set by means of an external device such as a smartphone provided with application software, e.g. a downloaded “app”, allowing a dose of drug to be set and transmitted wirelessly, e.g. by NFC or Bluetooth. In the shown embodiment a combined power and data communication port is provided, e.g. a USB port. The controller circuitry is further adapted to receive input from user input means (see below) as well as from one or more sensors, e.g. the shown piston rod sensor, just as the controller circuitry is adapted to control a display in accordance with detected operational conditions. The piston rod sensor may be used to detect piston rod engagement with the cartridge piston when the piston rod is forwarded after cartridge exchange, however, as will be described below, the piston rod sensor may also be used in embodiments of the present invention.

Turning to aspects of the present invention, the invention is based on a drug delivery device corresponding to the above-described generic type but provided with means for estimating the amount of free air in a loaded cartridge. Although the estimation of free air is not part of the present invention per se, examples of principles which may be used to provide the desired information will be described.

More specifically the drug delivery device 1 of FIG. 2 may be adapted to determine the amount of free air in the cartridge by incorporating means for measuring and acquiring a property relating to a fluid pressure of the drug in a received cartridge where the property depends on the amount of free air contained in the cartridge. The means for measuring this property may be the shown washer sensor 23 in the form of a force sensor or a proximity sensor. The electronic controller circuitry 70 may be provided with processor means for processing the acquired measurements during operation, and for estimating the amount of free air in the cartridge.

During operation the piston drive member is moved distally in order to pressurize the drug, the controller keeping track of the axial position. As air is compressible and drug is very little compressible it is possible to determine the amount of free air from the correlation between piston position and a response in the property related to the fluid pressure within the cartridge.

In a simple set-up free air is estimated when the distal outlet is closed as is the case when no needle assembly is mounted, whereas in a more advanced set-up free air can be estimated during operation when a needle assembly is mounted. A given device may be adapted to operate in one or two modes corresponding to the two conditions, e.g. by detecting the presence of a mounted needle.

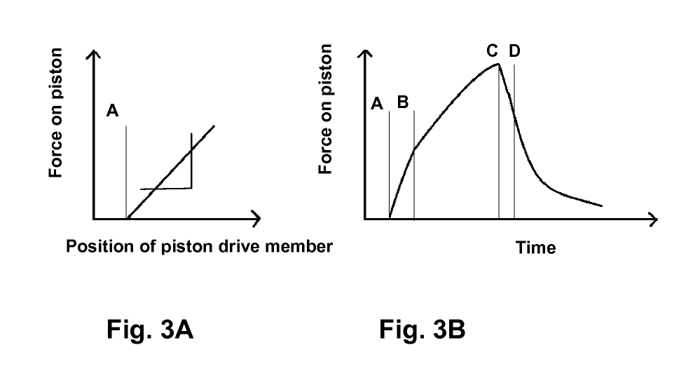

In the first mode it would be possible to use a simple method for determining the amount of air. FIG. 3A illustrates schematically the output of a measurement with a force sensor as a function of the position of the piston drive member, when the outlet portion is closed. The point A indicates contact between the piston drive member and the piston, and after this point there will be a linear relationship between the fluid pressure and the position of the piston drive member, with the assumption that the temperature is constant. After the point A any further advancement of the piston drive member will create a response in the fluid pressure. As the pressure and the position of the point A, and the pressure and the position after a further advancement of the piston drive member can be obtained, the amount or volume of free air at the point A, when no pressure is applied, can be determined.

FIG. 3B schematically illustrates the output of a measurement with a force sensor as a function of time, when the outlet portion is open. The sensor measuring the force between a piston drive member and a piston with a constant cross sectional area is easily related to the fluid pressure inside the cartridge. The general relation is that pressure equals force per unit area. The point A indicates contact between the piston drive member and the piston and also indicates the start of an acceleration phase, where the piston drive member accelerates the piston and pressurizes and expels the fluid. The point B indicates the end of the acceleration phase and the beginning of a phase wherein the piston drive member moves with a constant speed. The point C indicates the end of the constant speed phase and the beginning of a deceleration phase, where the fluid is still expelled although the fluid pressure decreases. The point D indicates the end of the deceleration phase, and the beginning of a relaxation phase wherein the piston drive member has stopped but wherein drug is still expelled due to relaxation of compliant parts, e.g. free air. If free air is present in the cartridge it will usually be the main contributing part to the overall compliance of the system but other components like piston, piston drive member and septum may also contribute. The relaxation phase ends when the fluid pressure is equal to the ambient pressure and no more drug is expelled. The acceleration phase, the constant speed phase and the deceleration phase are in common referred to as the stroke phase, as the piston drive member is advancing in all phases.

Different methods can be used to calculate the amount of free air based on measured pressure values. For example, calibration indicators calculated from calibration measurements may be stored in a memory, the processing means being configured for calculating a set of operation indicators and using the set of operation indicators and the set of calibration indicators to estimate the amount of free air in the cartridge during operation.

When using the drug delivery device several parameters can be varied by the user, e.g. the selected amount of drug to be expelled. Information of the selected parameters can be provided to the processor prior to dosing. Other variables like the amount of free air in the cartridge, different amounts of friction between piston and body portion of the cartridge, different type of flow conduits, i.e. different needles having different flow resistances, are typically unknown variables prior to dosing.

Therefore, the set of calibration indicators are calculated based on measurements of a property relating to the fluid pressure, for various selected amounts of drug to be delivered and optionally also one or more from the following group of parameters: dose delivery speed, different types of flow conduit, the amount of free air in the cartridge, different amounts of friction between piston and body portion of the cartridge. The set of calibration indicators are indicators used to characterize special features for the variation of the fluid pressure during the delivery of a dose for all possible conditions.

When a user starts delivery of a selected amount of drug, and when the drug delivery device is in the second mode, the processing means starts to process the measured and acquired measurements (e.g. a property relating to the fluid pressure, position of the piston drive member, time during operation), and applies an algorithm to calculate a new set of indicators, operation indicators, which comprises indicators used to characterize the fluid pressure during the current delivery. The set of operation indicators is continuously calculated and compared with the set of calibration indicators in order to estimate the amount of free air in the cartridge, and optionally also one or more of the following group of parameters: different types of flow conduit, different amounts of friction between piston and body portion of the cartridge

By this arrangement a set of stored calibration indicators enables a method of characterizing the response of the fluid pressure during delivery, and for estimating several unknown parameters during operation, by extracting a set of operation indicators and comparing the set with the set of calibration indicators. A given device is provided with the set of calibration indicators before the drug delivery device can be operational in the sense that it can be used for delivering an amount of drug to a patient. The set of calibration indicators can for example be provided before the drug delivery device is leaving production.

Alternatively the processing means may be configured for estimating the amount of free air in cartridge based on a physical model. Such an arrangement enables a method in where it is possible to estimate the amount of free air in a cartridge by the establishment of a physical model linking e.g. time during operation, the amount of free air in the cartridge, the amount of selected drug to be expelled, friction between piston and body portion of cartridge, and dose delivery speed. When estimating the amount of free air in the cartridge in the second mode it can be particularly useful to process data from the relaxation phase as the piston in this phase has stopped movement and there is consequently a minimal influence from the dynamic friction between the piston and the body portion of the cartridge. A more detailed description of concepts for determining the amount of free air in a cartridge can be found in patent application EP 2015/069889 which is hereby incorporated by reference.

Turning to aspects of the invention per se, a drug delivery device is provided (see FIGS. 1 and 2) comprising a cartridge holder for receiving a drug-filled cartridge in a loaded position, the cartridge comprising an outlet and an axially displaceable piston. The device comprises drug expelling means comprising dose setting means allowing a user to set a desired dose of drug to be expelled, a drive member adapted to engage and axially move the piston of a loaded cartridge to thereby expel an amount of drug from the cartridge through the outlet, and a motor for moving the drive member. An electronic controller is adapted to control the motor to move the drive member to thereby expel an amount of drug corresponding to a set dose. The device further comprises means for estimating the amount of free air in a loaded cartridge as discussed above. The controller is adapted to detect an error state if the estimated amount of free air in the cartridge is larger than or equals a given percentage of the amount of drug corresponding to the set dose. The drug delivery device may be provided with user alert means, e.g. audible, visual or tactile, the electronic controller being adapted to actuate the user alert means when an error state is detected.

In an exemplary embodiment the electronic controller is adapted to prevent a set dose of drug to be expelled when an error state is detected, however, the device may be provided with user input means allowing the user to override the electronic controller, e.g. by pushing a button for a given time, to thereby allow the motor to move the drive member corresponding to the set dose. Overriding the controller may be restricted to only some error conditions, e.g. if the amount of estimated free air is below a given amount. An error condition may also be removed by adjusting the set dose to an allowable dose size.

When treating type 2 diabetes it is considered a “major” failure if no insulin of a given dose is injected, however, it may be considered only a “minor” failure if at least some insulin of a given dose is injected. Indeed, this is not the case in the treatment of type 1 diabetes.

By this arrangement the traditional “air shot” step performed by the user to remove any air in the cartridge prior to injecting a dose of drug can be eliminated.

It should be emphasized that the presence of free air in a cartridge represents a risk that some or all of this air is expelled during a dosing event, and not that some or all of this free air necessarily will be expelled. In fact, in most cases it can be expected that no free air will be expelled during a dosing event. More specifically, during dose delivery it can be expected that most patients will hold the drug delivery device, which typically is pen-formed, at an inclined angle with the proximal end of the device oriented upwardly whereby the free air in the cartridge in most cases will flow to the proximal end of the cartridge opposite the distally arranged outlet. Indeed, if the cartridge is almost empty the free air may take up a relative large portion of the cartridge total remaining volume this increasing the likelihood of air being expelled. To take this into consideration the error conditions may be dynamic taking into account the percentage of total volume taken up by free air, see below.

As stated above, the controller is adapted to detect an error state if the estimated amount of free air in the cartridge is larger than or equals a given percentage of the amount of drug corresponding to the set dose. The percentage may be chosen according to the accepted risk of under-dosing.

For example, the percentage may be set to 50%. This means that if an amount of free air corresponding to 10 IU is estimated then an error is detected when a dose of at least 20 IU is set. If the percentage is set to 100% and an amount of free air corresponding to 10 IU is estimated then an error is detected when a dose of at least 10 IU is set.

To cope with the situation in which a relatively large amount of free air is present when the cartridge is almost empty the error condition may be adjusted, e.g. an error may be detected if a set dose is larger than the estimated amount of drug left in the cartridge.

For example, if the amount of drug left corresponding to the position of the piston is 30 IU and the estimated amount of free air corresponds to 10 IU then the estimated amount of drug left is 20 IU, this resulting in an error condition when a dose of at least 20 IU is set.

Depending on whether the drug delivery device is adapted to estimate the amount of free air with or without a needle assembly mounted, the use scenario for the device may differ.

For example, if the drug delivery device is provided with a simple estimating feature which only works when no needle assembly is mounted the device may be provided with a cap which does not allow the cap to be mounted when a needle assembly is mounted, this forcing the user to remove the needle assembly after use to allow the cap to be remounted. Correspondingly, when the user removes the cap the controller will automatically move the driver into contact with the piston (if a gap is present) and pressurize the cartridge a certain amount, at the same time measuring the relevant parameters. After the pressurization has been performed the drive member is retracted to reduce the pressure in the cartridge. Based on the measured values the amount of free air will be calculated. To cope with the situation in which a user leaves a needle assembly on, the device may be adapted to prevent setting a dose unless an estimate of free air has been performed just prior to setting a dose. Further, to cope with the situation in which a user mounts a needle immediately after having removed the cap and before the pressure measurements have been finalized, the controller may be adapted to detect such a situation, e.g. lack of pressure build-up.

Alternatively, if the drug delivery device is provided with a more advanced estimating feature which works during out-dosing the user can use the device without changes to the normal use procedure, the amount of free air being estimated during an out-dosing event, after which the estimate can be used to detect a potential error condition during a subsequent dose setting event.

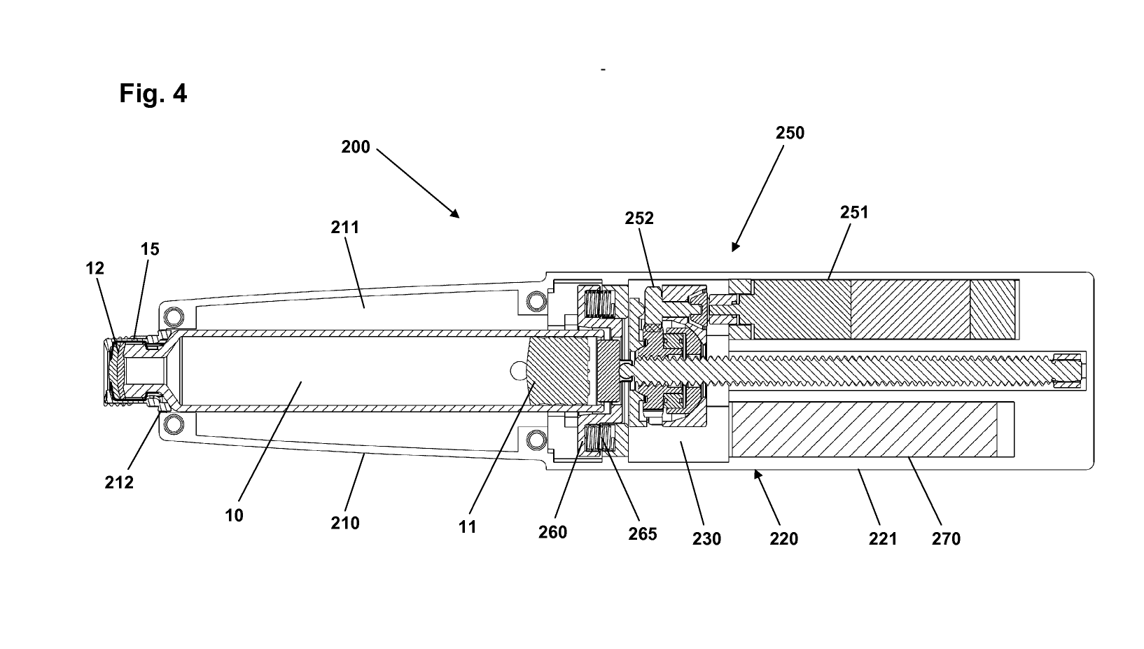

Turning to FIG. 4 a first embodiment 200 of a drug delivery device suitable as a platform for embodiments of the present invention will be described. More specifically, the device comprises a cap part (not shown) and a main part having a proximal body or drive assembly portion 220 with a housing 221 in which a motorized drug expelling assembly 250, electronic controller circuitry 270 and an electric power source are arranged, and a distal cartridge holder portion 210 with a compartment 211 in which a drug-filled cartridge 10 is arranged and retained in place. The cartridge comprises a generally cylindrical main portion with an axially displaceable piston 11 and a distal outlet portion 12 comprising a needle-penetrable septum. The cartridge is further provided with distal coupling means in the form of a needle hub mount 15 having, in the shown example, an external thread adapted to engage an inner thread of a corresponding hub of a needle assembly. The cartridge may for example contain an insulin, a GLP-1 or a growth hormone formulation. The device further comprises dose setting means allowing a user to set a dose of drug to be expelled as well as a display showing the set dose, e.g. as shown in FIG. 1.

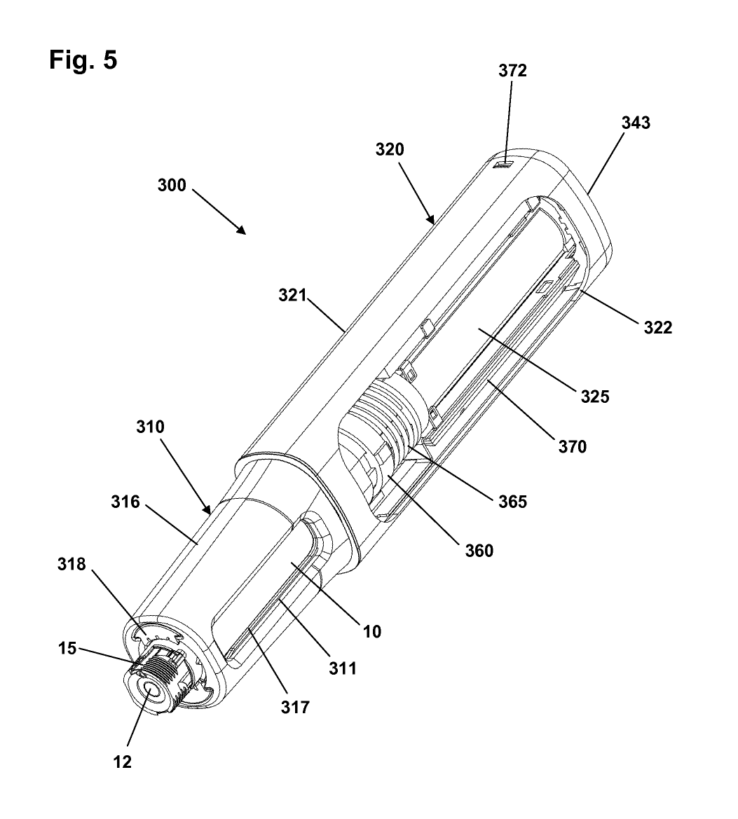

In the shown embodiment the device is designed to be loaded by the user with a new cartridge through a distal receiving opening 212 in the cartridge holder assembly, the cartridge holder comprising closure means (not shown) operatable by a user between an open position in which a cartridge can be inserted respectively removed, and a closed position in which an inserted cartridge is held in place. The closure means may be of the same type as described with respect to FIG. 5 below. In order to axially position the cartridge, the device comprises a seat member 260 adapted to receive the proximal end of the cartridge, the seat member being biased in the proximal direction by springs 265 thereby forcing the cartridge into contact with the closure means.

Turning to FIG. 5 a second embodiment 300 of a drug delivery device suitable as a platform for embodiments of the present invention will be described. More specifically, the device comprises a cap part (not shown) and a main part having a proximal body or drive assembly portion 320 with a housing 321 in which a drug expelling mechanism and associated electronics 370 are arranged, and a distal cartridge holder assembly 310 forming a compartment in which a drug-filled transparent cartridge 10 can be arranged and retained in place, the cartridge holder assembly comprising a pair of opposed inspection openings 311. The housing comprises an opening 322 adapted to receive a display frame member (not shown) in which a LCD as well as user input keys are mounted, e.g. as shown in FIG. 1. With the frame member removed, it can be seen that the device comprises a generally tubular chassis member 325, in which a generally cylindrical expelling assembly is mounted (see below). The device further comprises a control assembly 370, a bias assembly comprising a bias member 360 and a spring 365, and a proximal release button 343. An external charger can be received in female connector 372. The connector may e.g. be a micro USB connector which would also allow data to be transferred between the drug delivery device and an external device, e.g. a PC. A pair of dose setting input keys (not shown) serves to manually set a desired dose of drug shown in the LCD and which can then be expelled when the release button 90 is actuated. The device is designed to be loaded by the user with a new cartridge through a distal receiving opening in the cartridge holder assembly.

The cartridge 10 comprises a cylindrical body portion, a distal outlet portion 12 with a distal needle-penetrable septum, and an axially displaceable piston having a proximal surface allowing a piston driver forming part of the expelling mechanism (see below) to engage the piston. The cartridge may for example contain an insulin, a GLP-1 or a growth hormone formulation. The cartridge is provided with distal coupling means in the form of a needle hub mount 15 having, in the shown example, combined thread and bayonet coupling means, each being adapted to engage an inner thread or bayonet coupling means of a corresponding hub of a needle assembly. The shown exemplary hub mount further comprises a circumferential flange with a number of distally facing pointed projections serving as a coupling means for the cartridge holder assembly as will be described in more detail below. A hub mount of the shown type is described in U.S. Pat. No. 5,693,027. Alternatively the needle hub mount may be formed as part of the cartridge holder, e.g. in the form of a “split” hub mount having two parts arranged on each side of the gripping shoulders.

As shown, the cartridge holder assembly 310 has the same general appearance as a traditional cartridge holder which is detachably coupled to the housing by e.g. a threaded coupling or a bayonet coupling and into which a new cartridge can be received as well as removed through a proximal opening, i.e. it comprises no additional user operated release or locking means. Instead, what appears merely to be the cartridge holder per se is in fact user operated coupling means in the form of an outer rotatable tubular actuation sleeve 316 operated by the user to control movement of cartridge holding means in the form of an inner cartridge holder member 317 to thereby open and close gripping shoulders 318 configured to grip and hold a cartridge. More specifically, each gripping shoulder is provided with a plurality of gripping teeth spaced circumferentially to provide a plurality of gaps, each tooth having a triangular configuration with a proximally oriented pointed end, thereby creating a plurality of gaps having a distally oriented pointed configuration, this allowing the above-described distally facing pointed projections on the cartridge to be received between the teeth to thereby serve as a gripping means when the cartridge holding means has been moved into engagement with the cartridge. In this way an easy-to-use front loaded drug delivery device is provided which appears as a traditional rear loaded device and which is also actuated by rotational movement to mount and remove a cartridge, the resemblance providing for ease of acceptance and adaptation among users accustomed to traditional types of rear loaded drug delivery devices.

When it is time to mount a new cartridge the outer tube member 316 is rotated e.g. 90 degrees by which action the gripping shoulders 318 are moved distally and slightly outwards, this allowing the mounted cartridge to be removed. For ease of operation the cartridge may be moved distally a certain distance as the shoulders are moved, e.g. by engagement with arms forming the gripping shoulders and/or by additional spring means providing a biasing distally directed force (see below). Depending on the design of the locking and actuation mechanism the gripping shoulders may be able to be left in the open position or they may be retracted automatically as the outer tube member is rotated backwards by return spring means. Whether or not a spring is provided the cartridge holder may be provided with locking means allowing the outer tube member to be securely parked in either the open or closed position, e.g. by a rotational snap lock. When a new cartridge is inserted the drive expelling means has to be in a state allowing a new cartridge with a proximally positioned piston to be inserted. An exemplary embodiment providing this functionality will be described below.

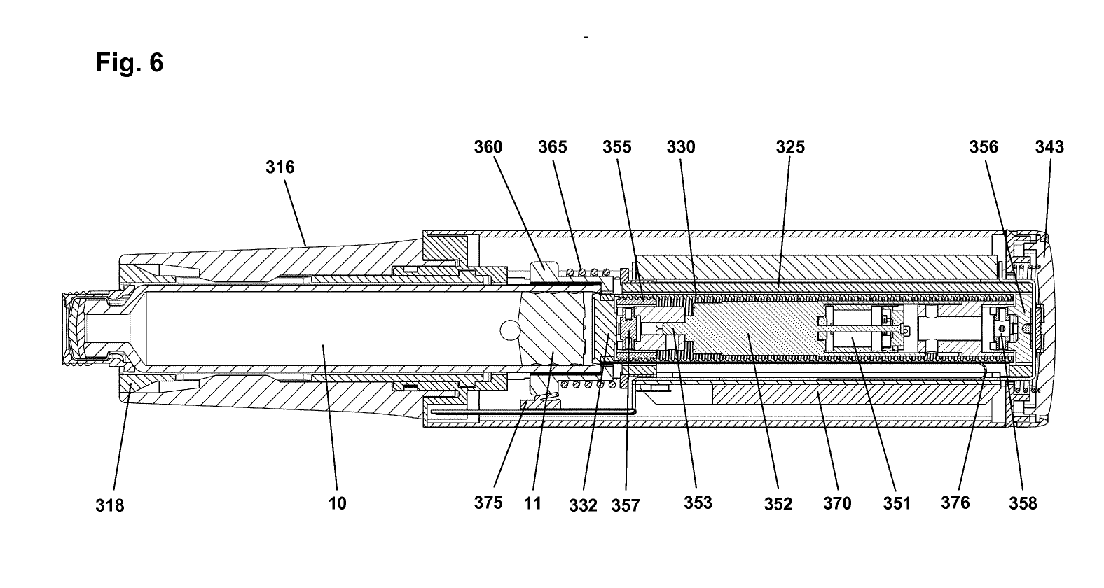

Turning to FIG. 6 a cross-sectional view of the drug delivery device 300 of FIG. 5 is shown with a mounted cartridge 10 and with the piston tube 330 (see below) in a fully retracted position. More specifically, the actuation sleeve 316 has been rotated to its operational position and the cartridge holder gripping shoulders 318 have been retracted to their closed position thereby retracting the cartridge to its fully inserted position, thereby also moving the bias member 360 proximally against the bias of the spring 365. In the shown embodiment a cartridge switch 375 is hereby being actuated, this providing a signal to the device controller that two actions can be assumed to have taken place: (i) a cartridge has been inserted and (ii) the cartridge holder has been closed, this initiating that the drive head is moved distally into contact with the cartridge piston. In the shown embodiment it is contemplated that detection of contact between the drive head and the piston is detected by electronic sensor means arranged in the drive head, e.g. using proximity detection as disclosed in WO 2013/144152.

FIG. 6 also shows the expelling assembly in greater detail. More specifically, the expelling assembly is in the form of a motor-in-piston assembly comprising an interior motor and gearbox drive assembly mounted axially and rotationally locked to the proximal end of the chassis, and an outer axially displaceable piston tube 330 with a distal drive head 332 adapted to engage the piston 11 of a loaded cartridge, the piston tube comprising a number of guide projections adapted to non-rotationally engage corresponding guide means of the chassis.

The motor-gear drive assembly comprises a tubular main portion 310 composed of a proximal motor assembly 351 and a distal gearbox assembly 352 having a rotatable drive shaft 353 defining a z-axis of rotation. The assembly further comprises a distal cylindrical drive member 355 having an outer thread adapted to be arranged in engagement with the piston drive tube inner thread. At the proximal end a disc-formed chassis connector 356 is arranged. In the shown embodiment the drive assembly is provided with flexible joints in the form of a distal universal joint 357 arranged between the drive shaft and the drive member and a proximal universal joint 358 arranged between the motor assembly proximal portion and the chassis tube proximal portion. A corresponding drive assembly is described in greater detail in patent application EP 14166859.0, which is hereby incorporated by reference.

A number of further details can be seen in FIG. 6. The release button 343 is received in the housings proximal opening with a spring providing a proximally directed biasing force on the button. A flexible ribbon 376 with a plurality of conductors is arranged with a U-bend between the electronics portion 370 and the sensors (not shown) arranged in the piston head, this allowing the piston tube and piston head to travel axially with the U-bend moving correspondingly.

In the above description of the preferred embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A drug delivery device, comprising:
a drug-filled cartridge or a compartment for receiving a drug-filled cartridge in a loaded position, the cartridge comprising an outlet and an axially displaceable piston,
a drug expelling mechanism comprising:
a drive member adapted to engage and axially move the piston of a loaded cartridge to thereby expel an amount of drug from the cartridge through the outlet, and
a motor for moving the drive member,
a dose setting structure allowing a user to set a desired dose of drug to be expelled,
an electronic controller adapted to control the motor to move the drive member to thereby expel an amount of drug corresponding to a set dose, and
a structure for estimating the amount of free air in a loaded cartridge,
wherein the electronic controller is further adapted to detect an error state if the estimated amount of free air in the cartridge is larger than or equals a given percentage of the amount of drug corresponding to a set dose.

2. A drug delivery device as in claim 1, further comprising a user alert structure, the electronic controller being adapted to actuate the user alert structure when an error state is detected.

3. A drug delivery device as in claim 1, wherein the electronic controller is adapted to prevent a set dose of drug to be expelled when an error state is detected.

4. A drug delivery device as in claim 3, further comprising a user input structure allowing the user to override the electronic controller to thereby allow the motor to move the drive member corresponding to the set dose.

5. A drug delivery device as in claim 1, comprising:
a structure for detecting a property value of the drug cartridge, the property value varying with the amount of free air contained in the cartridge, and
wherein the estimated amount of free air in the cartridge is based at least in part on the detected property value.

6. A drug delivery device as in claim 5, wherein the detected property value is related to the pressure in the cartridge.

7. A drug delivery device as in claim 5, further comprising a force sensor associated with the drive member, the force sensor providing an output related to the pressure in the cartridge during dose expelling.

8. A drug delivery device as in claim 5, wherein the cartridge piston is elastically deformable, the detected property value being related to deformation of the piston.

9. A drug delivery device as in claim 5, wherein the detected property value is the capacitance of the loaded cartridge or a portion of the loaded cartridge.

10. A drug delivery device as in claim 1, wherein the given percentage is settable.

11. A drug delivery device as in claim 1, wherein the electronic controller is further adapted to detect an error state if the estimated amount of drug in the cartridge is smaller than the amount of drug corresponding to the set dose, the estimated amount of drug in the cartridge taking into account the amount of estimated free air in the cartridge.

12. A drug delivery device as in claim 1, wherein:
the dose setting structure comprises communication allowing dose setting data to be received from an external device, and
the electronic controller is adapted to store received dose setting data and control the motor to move the drive member to thereby expel an amount of drug corresponding to a set dose.

13. A drug delivery device as in claim 12,
wherein the electronic controller comprises a clock and is adapted to store dose setting data representing at least two pre-set doses, each pre-set dose being associated with a time period of the day and/or week, and
wherein the controller is adapted to control the motor to move the drive member to thereby expel a stored dose amount according to the actual time.

14. A method of operating a drug delivery device, comprising the steps of:
providing a drug delivery device comprising:
a drug-filled cartridge comprising an outlet and an axially displaceable piston,
a drug expelling mechanism comprising:
a drive member adapted to engage and axially move the piston of a loaded cartridge to thereby expel an amount of drug from the cartridge through the outlet, and
a motor for moving the drive member,
a dose setting structure allowing a user to set a desired dose of drug to be expelled,
an electronic controller adapted to control the motor to move the drive member to thereby expel an amount of drug corresponding to a set dose, and
a structure for estimating the amount of free air in the cartridge,
estimating the amount of free air in the cartridge,
setting a dose to be expelled, and
detecting an error state if the estimated amount of free air in the cartridge is larger than or equals a given percentage of the amount of drug corresponding to the set dose.

15. A method of operating a drug delivery device as in claim 14, comprising the further step of:
when an error state is detected, actuating the user alert structure and/or preventing a set dose of drug to be expelled.
